(19) United States
(12) Patent Application Publication
(10) Pub. No.: US 2015/0238735 A1
(43) Pub. Date: Aug. 27, 2015

(54) GUIDE WIRE

(71) Applicant: ASAHI INTECC CO., LTD., Nagoya-shi (JP)

(72) Inventors: Muneya FURUKAWA, Seto-shi (JP); Kenta TSUGE, Nagakute-shi (JP)

(21) Appl. No.: 14/631,282

(22) Filed: Feb. 25, 2015

(30) Foreign Application Priority Data
Feb. 26, 2014 (JP) 2014-035222

(51) Int. Cl. A61M 25/09

(52) U.S. Cl. CPC A61M 25/09033; A61M 2025/09191

(57) ABSTRACT

A guide wire having sufficient flexibility and sufficient rigidity to prevent a bending point from progressing from a distal end of the guide wire to a proximal end of the guide wire even when the guide wire collides with a hard lesion. The guide wire includes a shaft and a first coil body wound around a distal portion of the shaft. The first coil body is formed by helically winding a stranded wire, which is formed by winding a plurality of element strands together. A joint portion is disposed at an intermediate part of the first coil body so as to join at least two adjacent stranded wires to each other without contacting the shaft.

### CROSS REFERENCE TO RELATED APPLICATION

This application claims priority to Japanese Application No. 2014-035222 filed on Feb. 26, 2014, the contents of which are incorporated by reference herein in their entirety.

### BACKGROUND

The disclosed embodiments relate to a medical device. Specifically, the disclosed embodiments relate to a guide wire to be inserted into a body cavity for medical treatment or examination.

Conventionally, various guide wires have been proposed to guide a catheter or the like into body tissues or through tubular organs such as blood vessels, digestive tracts, and ureters for medical treatment or examination. For example, Japanese Laid-Open Patent Publication No. 2007-90097 discloses a guide wire comprising a shaft and a coil that is wound around a distal portion of the shaft and is fixed to the shaft at an intermediate part by a fixing material. The shaft decreases in diameter toward its distal end.

When using a guide wire inside of blood vessels in a lower limb, sufficient flexibility is required at a distal portion of the guide wire in order to ensure that the guide wire can be directed through the intricately winding three-dimensional path of the blood vessels to reach the intended target. Moreover, if a distal end of the guide wire collides with a hard lesion during use of the guide wire in the lower limb, the distal portion may be bent. If one continues to use the guide wire in this state and inserts the guide wire further into the blood vessel, the bending can be worsened. That is, a bending point of the guide wire may shift from the distal portion to a proximal portion of the guide wire and may even reach a large diameter portion of the shaft. The large diameter portion of the shaft may become plastically deformed when bent, which makes it hard to recover the original shape of the distal portion by hand.

If the rigidity of a substantially intermediate portion of the guide wire is locally enhanced, the guide wire will bend at the high rigidity portion when the distal end of the guide wire collides with a hard lesion. As a result, the collision stress will be alleviated, and the bending point will not shift proximally beyond the high rigidity portion. Thus, plastic deformation of the large diameter portion of the shaft can be prevented.

However, in Japanese Laid-Open Patent Publication No. 2007-90097, since the intermediate part of the coil is fixed to the shaft by the fixing material, the rigidity at a fixing portion is locally enhanced, while the flexibility of the guide wire is diminished because the shaft is restrained by the fixing material. Although it may be possible to adjust the quantity of the fixing material in order to ensure the flexibility of the distal portion of the guide wire, such an adjustment may be difficult to implement and may not provide sufficient rigidity for the fixing portion. For this reason, if the distal end of the guide wire collides with a hard lesion, the bending point may shift from the distal portion to the proximal portion of the guide wire. That is, the guide wire disclosed in Japanese Laid-Open Patent Publication No. 2007-90097 still has room for improvement in that flexibility is not adequately balanced with the rigidity necessary to suppress the shifting of the bending point from the distal portion to the proximal portion even when the guide wire collides with a hard lesion.

### SUMMARY

From the viewpoint of the above circumstances, the disclosed embodiments were devised to provide a guide wire having both sufficient flexibility and rigidity to prevent a bending point from shifting from the distal portion to the proximal portion of the guide wire even when the guide wire collides with a hard lesion. In order to address the above-mentioned problems, the guide wire of the disclosed embodiments may include the following features.

A guide wire of the disclosed embodiments comprises a shaft and a first coil body wound around a distal portion of the shaft. The first coil body may be formed by helically winding a stranded wire, which is formed by winding a plurality of element strands together. A joint portion is inserted between the element strands and is disposed at an intermediate part of the first coil body so as to join together adjacent stranded wires without contacting the shaft.

When the joint portion is disposed on the first coil body formed by winding the stranded wire, a material forming the joint portion (hereinafter referred to as “joint material”) permeates into gaps between the element strands preferentially along the longitudinal direction due to capillary action. Thus, the rigidity at the joint portion is enhanced. At the same time, it is relatively difficult for the joint material to permeate in the direction orthogonal to the longitudinal direction (the direction extending radially toward the shaft). Accordingly, the joint portion is not connected to the shaft, and the shaft is not restrained by the joint portion.

Consequently, the flexibility at the distal portion of the guide wire is ensured without requiring complicated measures of adjusting the appropriate quantity of the joint material, and sufficient rigidity is also ensured by allowing the joint material to be inserted between the element strands along the longitudinal direction, thereby joining adjacent stranded wires to each other.

Therefore, a guide wire of the disclosed embodiments can be easily directed through an intricately winding three-dimensional path of a blood vessel (for example, in the lower limb), and yet when the distal end of the guide wire collides with a hard lesion, the collision stress is concentrated at the intermediate part of the first coil body where the rigidity is enhanced by the joint portion. The guide wire thus bends at the intermediate part in substantially a V-shape so that the stress is diminished and does not progress proximally beyond the intermediate part. As a result, even when the distal end of the guide wire collides with a hard lesion, a bending point of the guide wire does not shift to a large diameter section of the shaft, and plastic deformation of the shaft is prevented. The guide wire can therefore continue to be used.

The first coil body of the guide wire can alternatively be formed by helically winding a plurality of the stranded wires (where each stranded wire is formed by winding a plurality of element strands together). In this manner, the gap between the element strands becomes smaller by allowing the stranded wires to be in close contact with each other. Moreover, when the first coil body is formed from a plurality of stranded wires, the permeation of the joint material into gaps between the element strands along the longitudinal direction is facilitated. Thus, the rigidity at the joint portion is reliably enhanced. On the other hand, it becomes even more difficult for the joint material to permeate in the direction orthogonal to the longitudinal direction. Accordingly, the joint portion is not connected to the shaft, and the shaft is not restrained by the joint portion.

In the disclosed embodiments, a second coil body may be disposed inside the first coil body, and the joint portion may join both the first coil body and the second coil body without contacting the shaft. The second coil body may be formed by winding a single element strand, for example. The permeation of the joint material into the gaps between the element strands constituting the first coil body is therefore facilitated due to capillary action as described above, while the permeation of the joint material in the direction orthogonal to the longitudinal direction is blocked by the second coil body. Permeation of the joint material to the shaft is therefore effectively suppressed. The addition of the second coil body can thus simultaneously ensure the rigidity at the joint portion and suppress the permeation of the joint material to the shaft, ensuring sufficient flexibility without restraining the shaft by the joint portion.

The second coil body of the guide wire may alternatively be formed by helically winding a plurality of element strands together. When the joint portion is disposed in the intermediate part of the first coil body, the joint material permeates into the gaps between the element strands in both the first coil body and the second coil body preferentially along the longitudinal direction due to capillary action. Rigidity is therefore enhanced. At the same time, the permeation of the joint material in the direction orthogonal to the longitudinal direction is further blocked by the second coil body, preventing the joint material from reaching the shaft. The second coil body can therefore ensure both sufficient rigidity at the joint portion and sufficient flexibility.

The second coil body may also be formed by helically winding a plurality of the stranded wires (where each stranded wire is formed by winding a plurality of element strands together). When the second coil body has this structure, the gap between the element strands is even smaller because the stranded wires are in close contact with each other. As a result, when the joint portion is disposed at the intermediate part of the first coil body, the joint material further permeates into the gaps between the element strands of the second coil body along the longitudinal direction, and the rigidity at the joint portion is further enhanced. However, the permeation of the joint portion in the direction orthogonal to the longitudinal direction is still blocked by the second coil body.

The guide wires of the disclosed embodiments can therefore reliably ensure the rigidity at the joint portion without restraining the shaft by the joint portion. Thus, both sufficient rigidity and sufficient flexibility are ensured. That is, the guide wires of the disclosed embodiments can be easily directed through an intricately winding three-dimensional path of a blood vessel, and even if the distal end of the guide wire collides with a hard lesion, the collision stress is reliably concentrated at the intermediate part of the first coil body where the rigidity is enhanced by the joint portion. The stress is thus alleviated by the guide wire bending in substantially a V-shape at the intermediate part where the rigidity is locally enhanced, and the stress does not progress proximally beyond the intermediate part of the first coil body. Plastic deformation of the shaft is therefore more reliably suppressed.

### BRIEF DESCRIPTION OF THE DRAWINGS

FIG. 1 is a cross-sectional view of a portion of a guide wire of the disclosed embodiments.

FIG. 2 is a perspective view of a stranded wire of a first coil body of the guide wire of FIG. 1.

FIG. 3 is a cross-sectional view of a joint portion of the guide wire of FIG. 1.

FIG. 4 is a cross-sectional view of a portion of a guide wire of the disclosed embodiments in a bent condition.

FIG. 5 is a cross-sectional view of a portion of another guide wire of the disclosed embodiments.

FIG. 6 is a cross-sectional view of a portion of another guide wire of the disclosed embodiments.

FIG. 7 is a cross-sectional view taken along line A-A of FIG. 6.

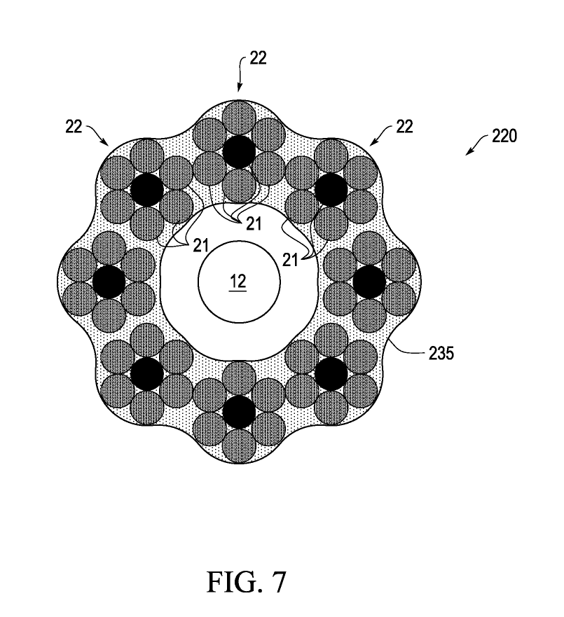

FIG. 8 is a cross-sectional view of a joint portion of the guide wire of FIG. 6.

FIG. 9 is a cross-sectional view of a portion of the guide wire of FIG. 6 in a bent condition.

FIG. 10 is a cross-sectional view of a portion of a guide wire of the disclosed embodiments.

FIG. 11 is a cross-sectional view of a portion of the guide wire of FIG. 10 in a bent condition.

FIG. 12 is a cross-sectional view of a portion of another guide wire of the disclosed embodiments.

FIG. 13 is a perspective view of a second coil body in the guide wire of FIG. 12.

FIG. 14 is a cross-sectional view of a joint portion of the guide wire of FIG. 12.

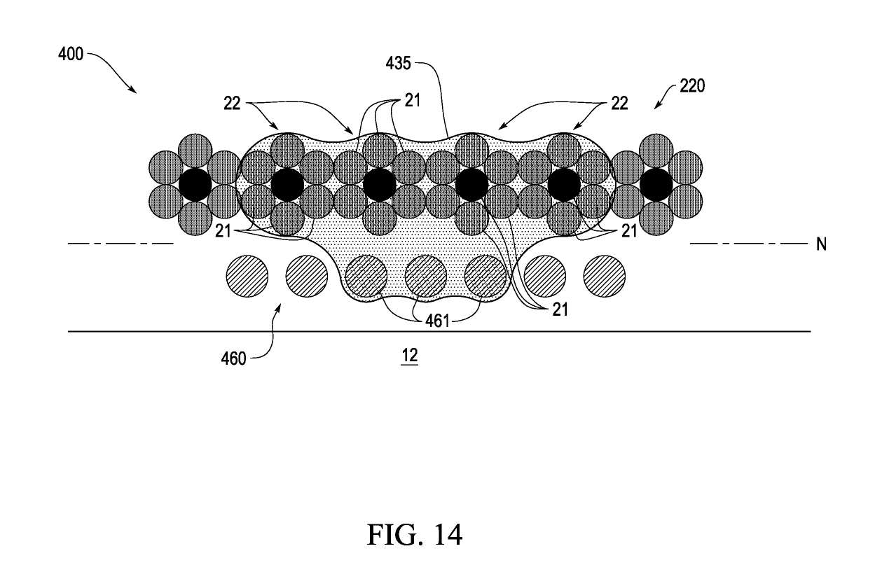

FIG. 15 is a cross-sectional view of a portion of the guide wire of FIG. 12 in a bent condition.

FIG. 16 is a cross-sectional view of a portion of a guide wire of the disclosed embodiments.

FIG. 17 is a cross-sectional view taken along line B-B of FIG. 16.

FIG. 18 is a cross-sectional view of a portion of the guide wire of FIG. 16 in a bent condition.

### DETAILED DESCRIPTION OF EMBODIMENTS

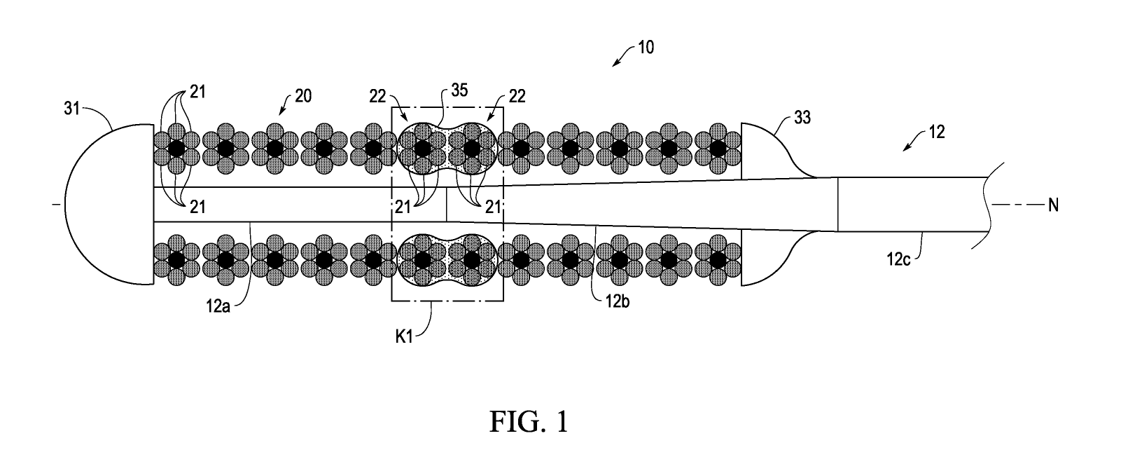

The guide wire of the disclosed embodiments is explained with reference to the drawings. In FIGS. 1, 4, 5, 6, 9, 10, 11, 12, 15, 16, and 18, the left side corresponds to a distal end of the guide wire to be inserted into a body, and the right side corresponds to a proximal end of the guide wire to be manipulated by a doctor or other technician. The drawings are not necessarily drawn to scale.

FIG. 1 is a cross-sectional view of a portion of a guide wire 10 of the disclosed embodiments. A guide wire 10 as illustrated in FIG. 1 may be used for medical treatment of lower limb blood vessels according to the Cross Over method. The guide wire 10 comprises a shaft 12 and a first coil body 20 covering the periphery of a distal portion of the shaft 12.

Firstly, the shaft 12 is explained. The shaft 12 includes a thin diameter section 12a, a tapered section 12b, and a large diameter section 12c in this order from a distal end to a proximal end of the shaft 12. The thin diameter section 12a is disposed at the distal end of the shaft 12 and is the most flexible portion of the shaft 12. The thin diameter section 12a is pressed and formed in a tabular shape. The tapered section 12b has a tapered shape with a round cross-section, and decreases in diameter toward the distal end. The large diameter section 12c has a larger diameter than the thin diameter section 12a. The material used for forming the shaft 12 may be, for example, stainless steel (SUS304), a superelastic alloy such as a Ni—Ti alloy, piano wire, or a cobalt alloy. However, the shaft 12 is not limited to these materials.

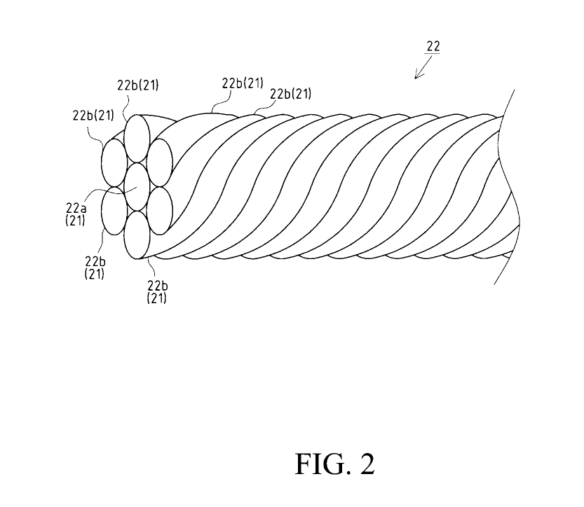

Next, the first coil body 20 is explained. In the guide wire of FIG. 1, first coil body 20 is formed by helically winding a stranded wire 22, which in turn is formed by winding a plurality of element strands 21 together (see FIG. 2). The stranded wire 22 includes a core element strand 22a and six side element strands 22b wound about the periphery of the core element strand 22a so as to cover the core element strand 22a. In FIG. 1, a pitch of a helix of the first coil body 20 is evenly arranged in the longitudinal direction “N.” The material used for forming the core element strand 22a and the side element strands 22b may be, for example, a stainless steel such as martensitic stainless, ferritic stainless, austenitic stainless, austenitic/ferritic two-phase stainless, or precipitation-hardened stainless; a superelastic alloy such as a Ni—Ti alloy; or a roentgenopaque metal such as platinum, gold, tungsten, tantalum, iridium, or alloys thereof. However, the element strands 21 are not limited to these materials.

A distal end of the first coil body 20 is fixed to the distal end of the shaft 12 by a distal fixing portion 31. A proximal end of the first coil body 20 is fixed to the shaft 12 by a proximal fixing portion 33. Moreover, a joint portion 35 is disposed at an intermediate part of the first coil body 20. A material used for forming the distal fixing portion 31, the proximal fixing portion 33, or the joint portion 35 may be, for example, a brazing metal such as a Sn—Pb alloy, Pb—Ag alloy, Sn—Ag alloy, or Au—Sn alloy. However, the distal fixing portion 31, proximal fixing portion 33, and joint portion 35 are not limited to these materials.

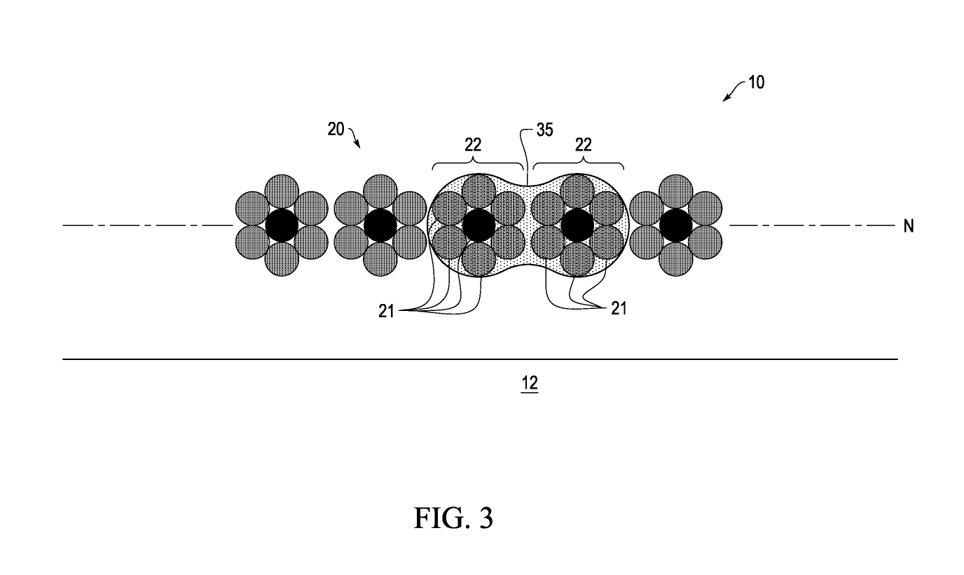

As shown in FIGS. 1 and 3, a material forming the joint portion 35 (the “joint material”) is inserted between the element strands 21 of the stranded wire 22 that forms the first coil body 20, and the joint portion 35 joins at least one pair of adjacent stranded wires 22 without connecting to the shaft 12.

Because the joint portion 35 is disposed in the first coil body 20 formed by winding the stranded wire 22, the joint material for forming the joint portion 35 permeates into gaps between the element strands 21 preferentially along the longitudinal direction “N” due to capillary action, thus enhancing the rigidity of the guide wire 10 at the joint portion 35. However, it is relatively difficult for the joint material to permeate in the direction orthogonal to the longitudinal direction “N” (the direction extending radially toward the shaft 12). Thus, the joint portion 35 is not connected to the shaft 12, and the shaft 12 is not restrained by the joint portion 35.

Consequently, the flexibility of a distal portion of the guide wire 10 is ensured without requiring complicated adjustments to the quantity of the joint material. Additionally, sufficient rigidity is ensured by allowing the joint material to permeate between the element strands 21 due to capillary action, thereby joining adjacent stranded wires 22.

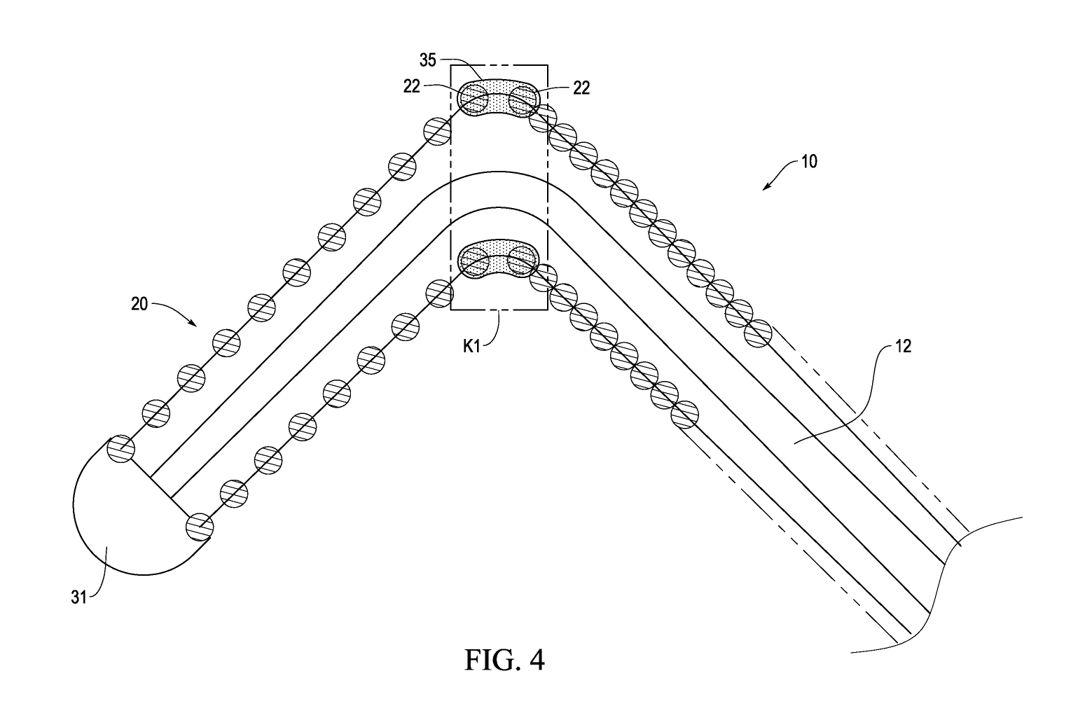

The guide wire 10 can therefore be easily directed through intricately winding and three-dimensional blood vessels (for example, in the lower limb), and even when the distal end of the guide wire 10 collides with a hard lesion, the collision stress is concentrated at the intermediate part of the first coil body 20 (the part K1 at which the rigidity is enhanced by the joint portion 35). The stress is thus alleviated by the guide wire 10 being bent in substantially a V-shape as in FIG. 4, and the stress does not progress proximally beyond the intermediate part of the first coil body 20. As a result, even if the distal end of the guide wire 10 collides with a hard lesion, the bending point will not shift to the large diameter section 12c, and plastic deformation of the shaft 12 is prevented. The guide wire 10 can therefore be used continuously. For simplicity, the detailed structure of the stranded wire 22 is not shown in FIG. 4.

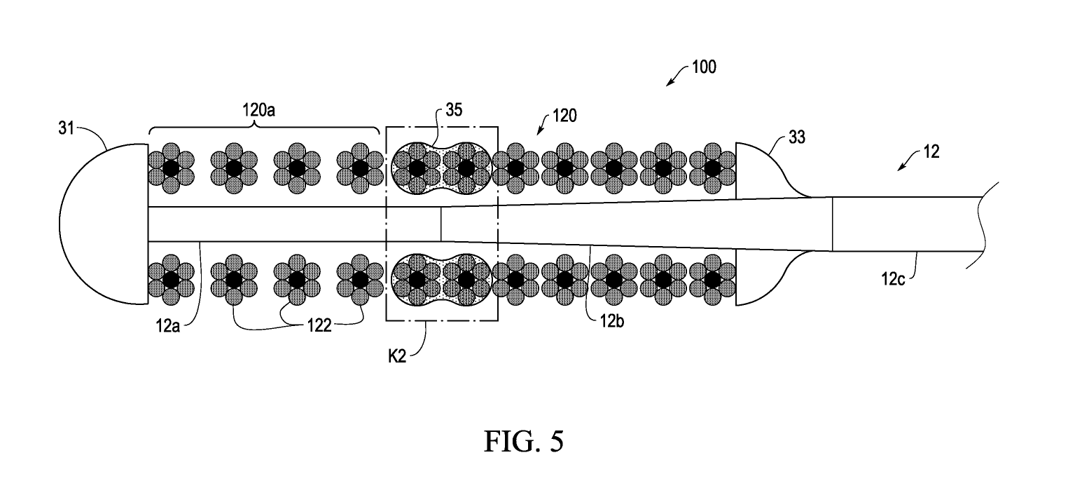

Although the pitch of the helix of the first coil body 20 of the embodiment may be evenly arranged in the longitudinal direction “N,” the helical condition of the first coil body 20 is not limited to this. That is, as shown in FIG. 5, a sparse winding section 120a may be disposed at a portion of the first coil body 120 extending distally beyond the joint portion 135. The sparse winding section 120a has a gap between adjacent stranded wires 122 that is larger than a gap between adjacent stranded wires 122 in a portion of the first coil body 120 proximal to the joint portion 135.

The flexibility at a distal portion of the guide wire 100 is therefore further improved, further facilitating maneuverability of the guide wire 100 through tortuous blood vessels. The rigidity of the guide wire 100 is sharply enhanced by the joint portion 135. As a result, when the distal end of the guide wire 100 collides with a hard lesion, the collision stress is likely to concentrate at the intermediate part of the first coil body 120 (the part K2 at which the rigidity is enhanced by the joint portion 135), and the stress is effectively alleviated by the guide wide bending in substantially a V-shape.

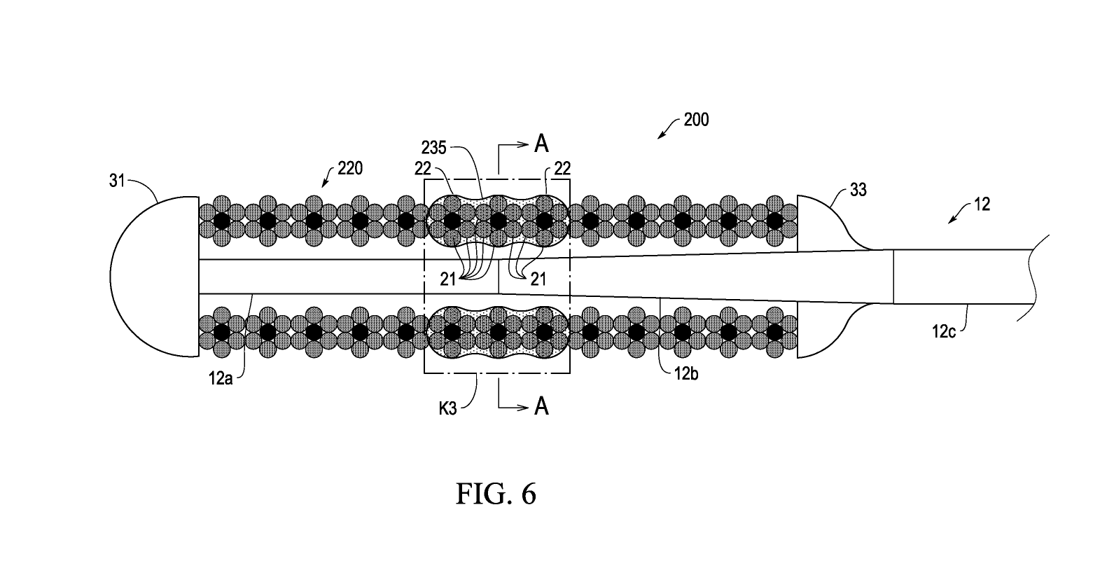

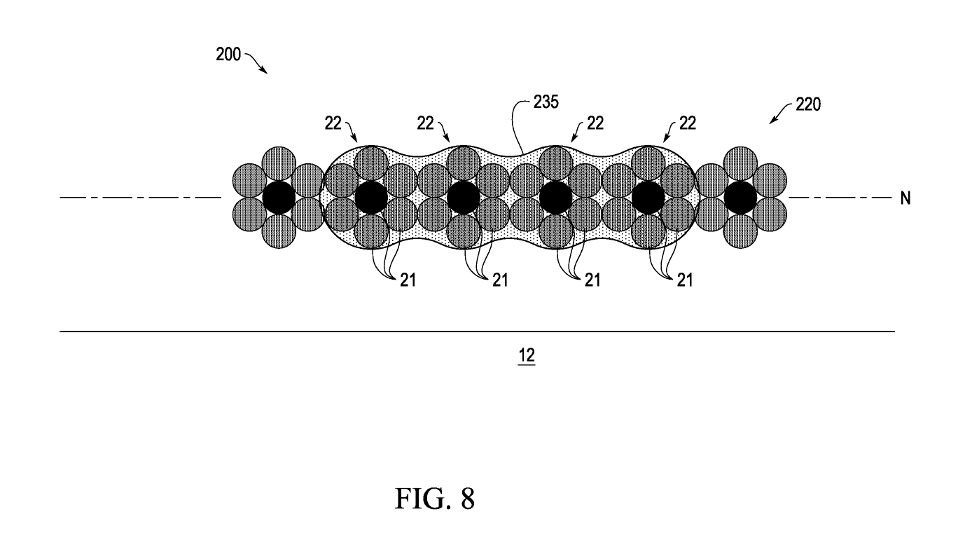

FIG. 6 is a cross-sectional view of a guide wire 200 of the disclosed embodiments. A first coil body 220 of the guide wire 200 is formed by helically winding a plurality of the stranded wires 22 (e.g., eight stranded wires 22). The stranded wires 22 are in close contact with each other, decreasing the size of the gaps between the element strands 21. As a result, the permeation of the joint material into the gaps between the element strands 21 is further facilitated along the longitudinal direction “N” (see FIG. 8), thus reliably enhancing the rigidity at the joint portion 235. At the same time, it becomes even more difficult for the joint material to permeate in the direction orthogonal to the longitudinal direction “N.” Accordingly, the joint portion 235 is not connected to the shaft 12, and the shaft 12 is not restrained by the joint portion 235.

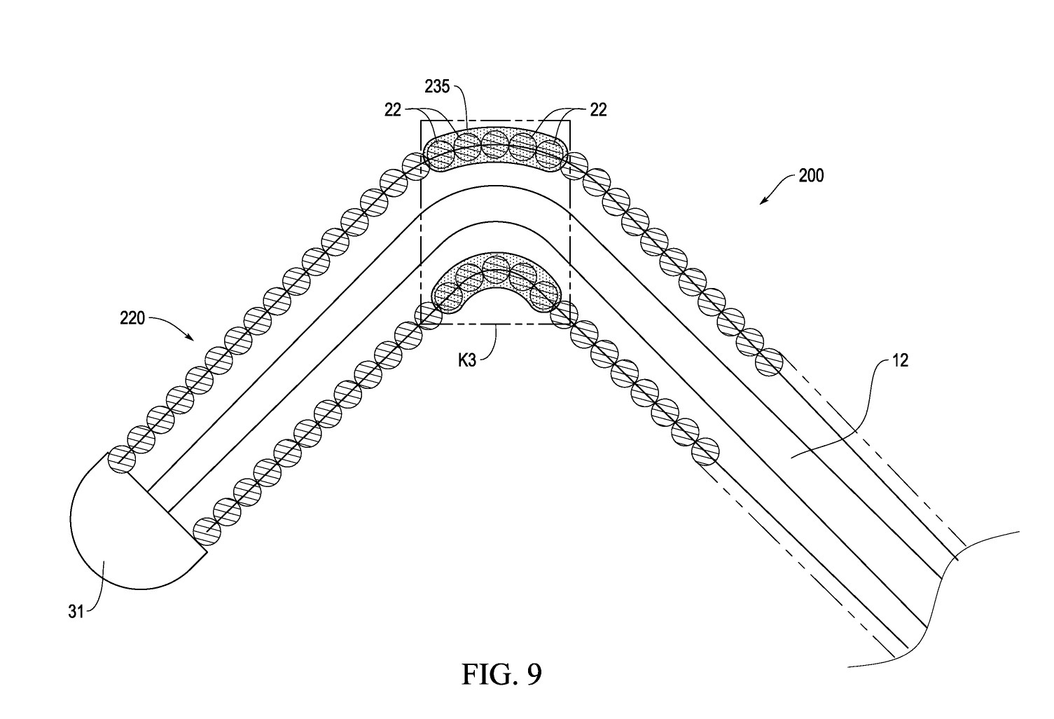

The guide wire 200 can therefore be easily directed through tortuous blood vessels, and collision stress is concentrated at the intermediate part of the first coil body 220 (the part K3 at which the rigidity is enhanced by the joint portion 235) even if the distal end of the guide wire 200 collides with a hard lesion. As a result, the stress is alleviated by allowing the guide wire 200 to bend in substantially a V-shape as shown in FIG. 9 at the part K3 at which the rigidity is enhanced by the joint portion 235. Because the stress does not progress proximally beyond the intermediate part of the first coil body 220, plastic deformation of the shaft 12 can be reliably suppressed. For simplicity, the detailed structure of the stranded wires 22 is not shown in FIG. 9.

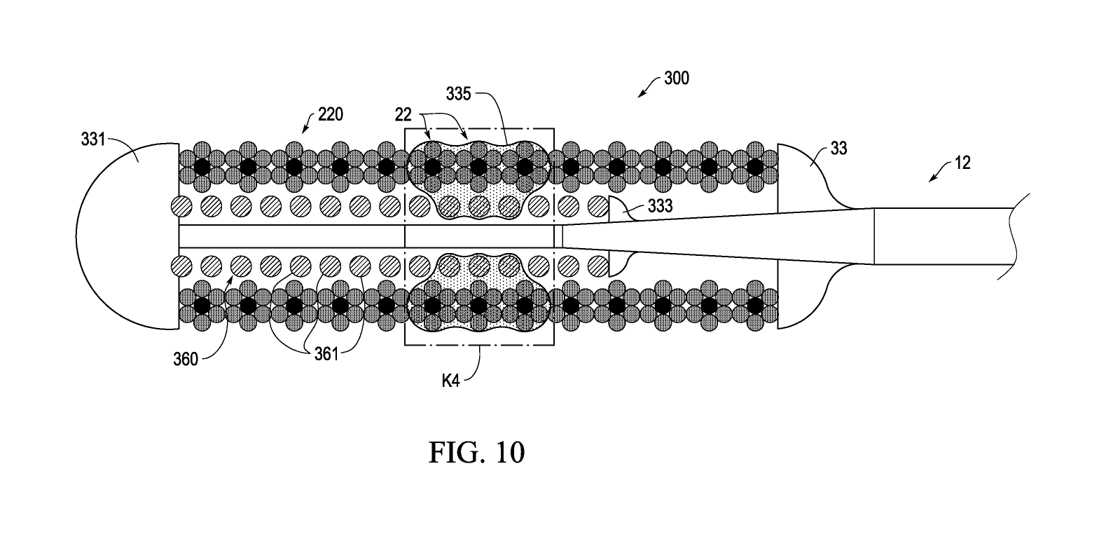

FIG. 10 is a cross-sectional view of a guide wire 300 of the disclosed embodiments. A second coil body 360 of the guide wire 300 is disposed inside of the first coil body 220 that is formed by helically winding a plurality of the stranded wires 22. The second coil body 360 is a single strand coil formed by helically winding one element strand 361. The second coil body 360 may be formed by a radiopaque element strand or a radiolucent element strand. A material used for a radiopaque element strand may be, for example, gold, platinum, tungsten, or an alloy of these elements (e.g., a platinum-nickel alloy). A material used for a radiolucent strand may be, for example, stainless steel (SUS304, SUS316, or the like), a superelastic alloy such as a Ni—Ti alloy, or piano wire. However, the second coil body 360 is not limited to these materials.

A distal end of the second coil body 360 is connected to the distal end of the shaft 12 by a distal fixing portion 331. A proximal end of the second coil body 360 is connected to the shaft 12 by an intermediate fixing portion 333. The material used for forming the intermediate fixing portion 333 may be, for example, a brazing metal such as a Sn—Pb alloy, Pb—Ag alloy, Sn—Ag alloy, or Au—Sn alloy. However, the intermediate fixing portion 333 is not limited to these materials.

A joint portion 335 disposed at the intermediate part of the first coil body 220 joins the first coil body 220 and the second coil body 360 without contacting the shaft 12. The permeation of the joint material into the gaps between the element strands 21 of the first coil body 220 is facilitated due to capillary action. Meanwhile, the second coil body 360 prevents the joint material from permeating in the direction orthogonal to the longitudinal direction “N” and reaching the shaft 12. Therefore, the rigidity at the joint portion 335 is ensured without restraining the shaft 12, thus also ensuring sufficient flexibility.

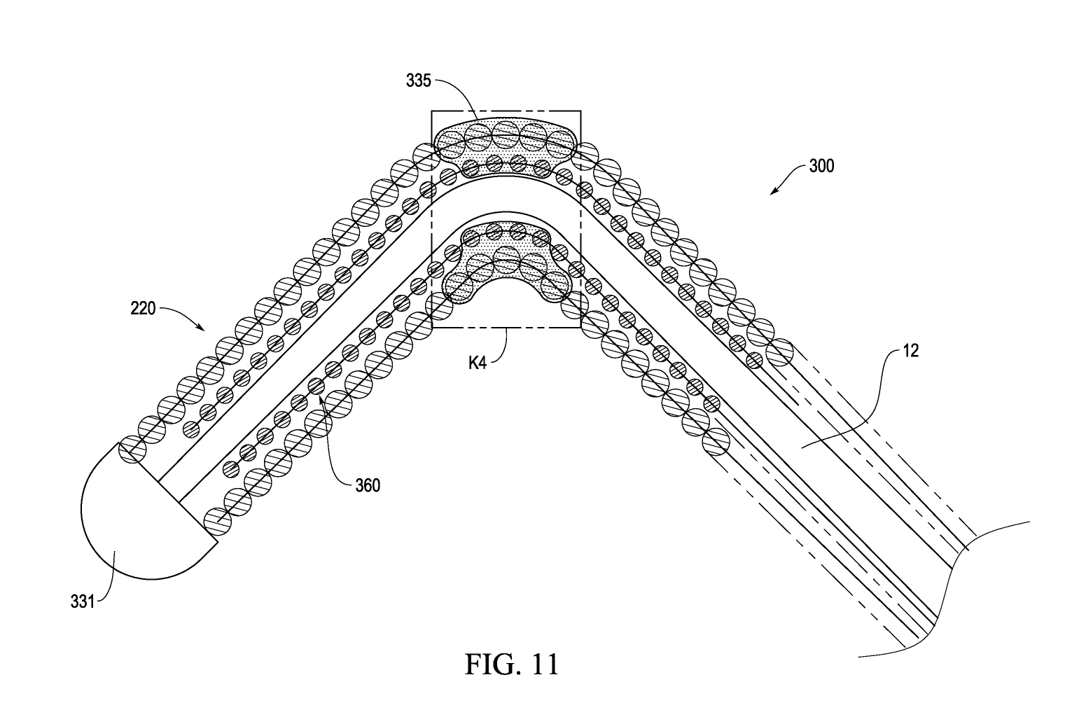

That is, the guide wire 300 also can be easily directed through tortuous blood vessels, and even if the distal end of the guide wire 300 collides with a hard lesion, the guide wire 300 can alleviate the collision stress by allowing the stress to easily concentrate at the intermediate part of the coil body 220 (the part K4 at which the rigidity is enhanced by the joint portion 335) and by allowing the guide wire 300 to bend in substantially a V-shape (see FIG. 11). The stress does not progress proximally beyond the intermediate part of the first coil body 220, and plastic deformation of the shaft 12 can be suppressed effectively. For simplicity, the detailed structure of the stranded wires 22 is not shown in FIG. 11.

In FIG. 10, the first coil body 220 is formed by helically winding a plurality of the stranded wires 22. However, the first coil body 220 may be formed by helically winding one stranded wire 22. Also in this case, the rigidity of the joint portion 335 can be easily enhanced by allowing the joint material to preferentially permeate in the longitudinal direction due to capillary action.

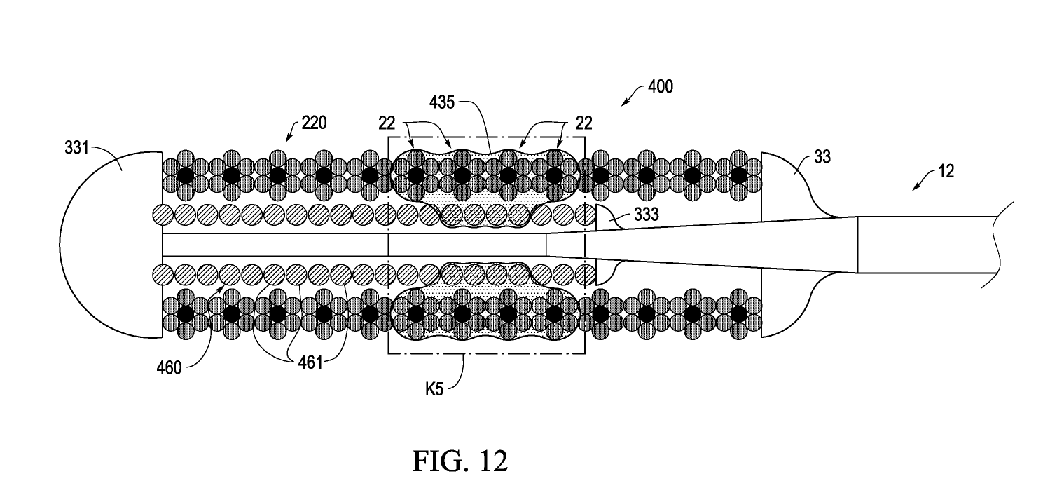

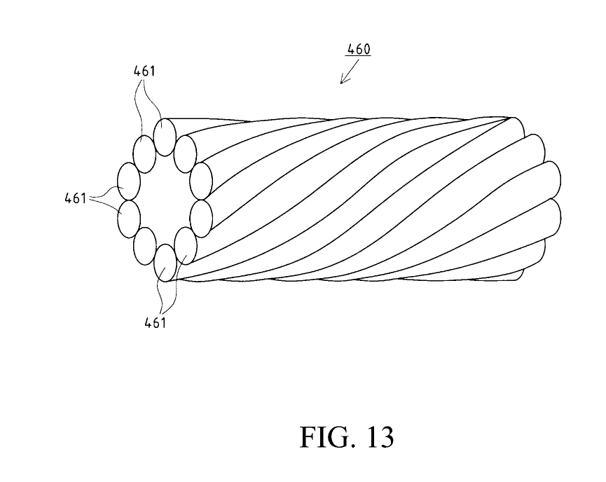

FIG. 12 is a cross-sectional view of a guide wire 400 of the disclosed embodiments. As shown in FIG. 13, a second coil body 460 is formed by winding a plurality of element strands 461 (e.g., ten element strands 461). In the guide wire 400, a joint portion 435 is disposed in both the intermediate part of the first coil body 220 and in the second coil body 460 that is formed by winding a plurality of the element strands 461 together. The joint material therefore permeates into the gaps between the element strands 461 preferentially along the longitudinal direction “N” due to capillary action so that the rigidity is enhanced. That is, in the guide wire 400, the joint material permeates into the gaps between the element strands 21 that form the first coil body 220 and the gaps between the element strands 461 that form the second coil body 460 along the longitudinal direction “N.” On the other hand, the permeation of the joint material in the direction orthogonal to the longitudinal direction “N” and toward the shaft 12 is blocked by the second coil body 460. Therefore, in the guide wire 400, the permeation of the joint material of the joint portion 435 in the longitudinal direction “N” with respect to the first coil body 220 and the second coil body 460 can ensure sufficient rigidity as well as sufficient flexibility without restraining the shaft 12 by the joint portion 435.

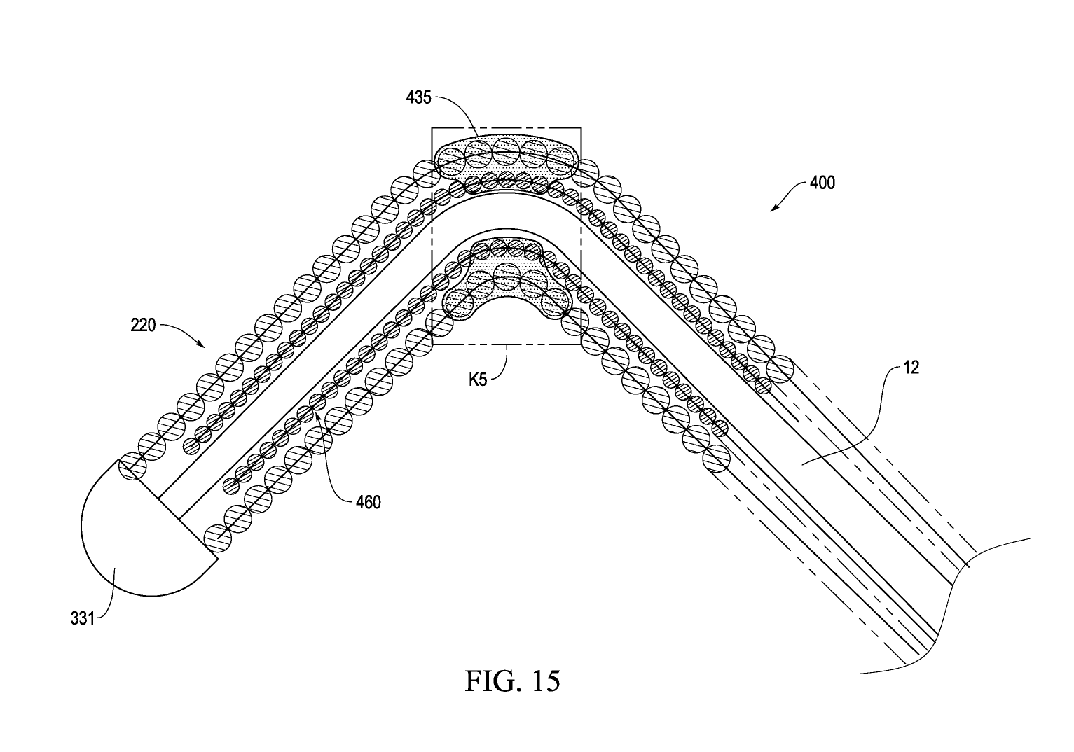

That is, the guide wire 400 also can be easily directed through tortuous blood vessels, and even if the distal end of the guide wire 400 collides with a hard lesion, the guide wire can alleviate the collision stress by allowing the stress to reliably concentrate at the intermediate part of the coil body 220 (the part K5 at which the rigidity is enhanced by the joint portion 435) and by allowing the guide wire 400 to bend in substantially a V-shape (see FIG. 15). The stress does not progress proximally beyond the intermediate part of the first coil body 220, and plastic deformation of the shaft 12 can be suppressed reliably. For simplicity, the detailed structure of the stranded wires 22 is not shown in FIG. 15.

In FIG. 13, the second coil body 460 is formed by winding a plurality of the element strands 461 together. However, the constitution of the second coil body 460 is not limited to this. That is, the second coil body 460 may be formed by helically winding a stranded wire containing a core element strand and side element strands wound about and covering the periphery of the core element strand as in the stranded wire 22 shown in FIG. 2. Also in this case, the rigidity of the joint portion 435 can be easily enhanced by allowing the joint material to preferentially permeate along the longitudinal direction due to capillary action.

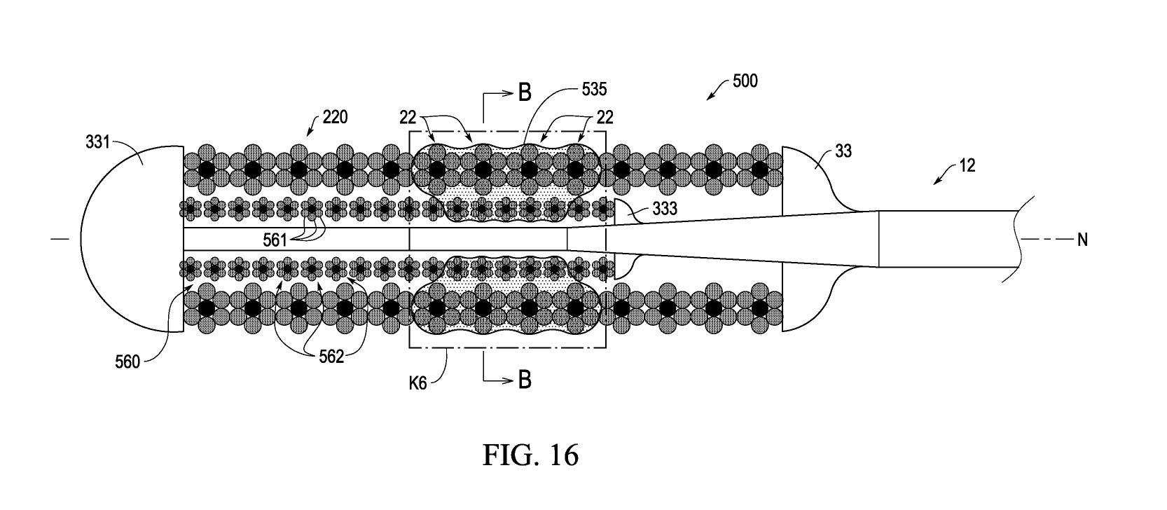

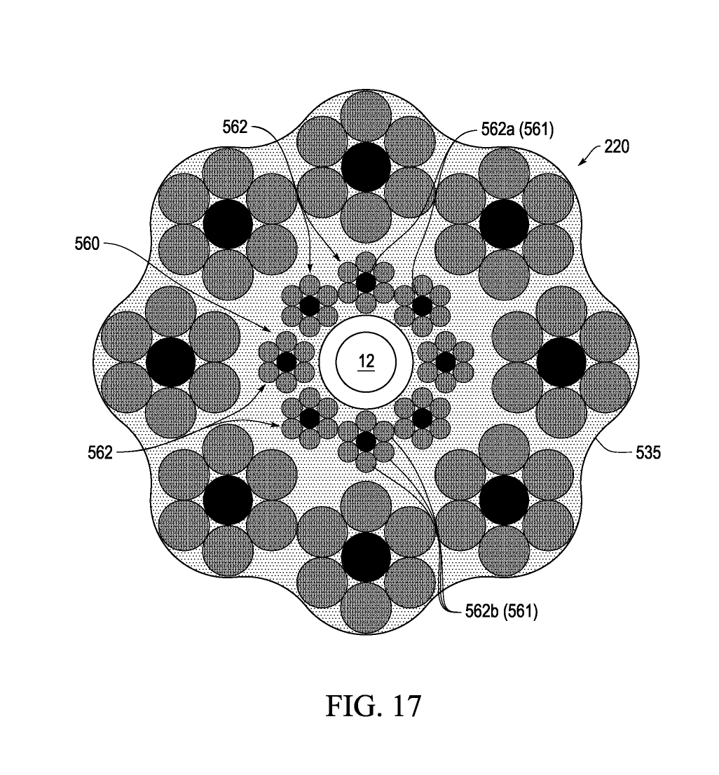

FIG. 16 is a cross-sectional view of a guide wire 500 of the disclosed embodiments. As shown in FIGS. 16 and 17, a second coil body 560 of the guide wire 500 is formed by helically winding a plurality of stranded wires 562 (e.g., eight stranded wires 22). Each stranded wire 562 is formed by winding a plurality of element strands 561 together. More specifically, as shown in FIG. 17, the second coil body 560 is formed by helically winding the eight stranded wires 562, each stranded wire 562 including a core element strand 562a and six side element strands 562b wound about and covering the periphery of the core element strand 562a. The material used for forming the core element strand 562a and side element strands 562b for the second coil body 560 may be, for example, a stainless steel such as martensitic stainless, ferritic stainless, austenitic stainless, austenitic/ferritic two-phase stainless, or precipitation-hardened stainless; a superelastic alloy such as a Ni—Ti alloy; or a roentgenopaque metal such as platinum, gold, tungsten, tantalum, iridium, or alloys thereof.

The gaps between the element strands 561 become smaller by allowing the stranded wires 562 to be in closer contact with each other. As a result, by disposing the joint portion 535 at the intermediate part of the first coil body 220, the permeation of the joint material along the longitudinal direction “N” into the gaps between the element strands 561 that form the second coil body 560 is further facilitated, thus enhancing the rigidity of the guide wire 500 at the joint portion. On the other hand, the permeation of the joint material in the direction orthogonal to the longitudinal direction “N” toward the shaft 12 is blocked by the second coil body 560. Therefore, rigidity at the joint portion 535 is ensured without restraining the shaft 12, thus also ensuring sufficient flexibility.

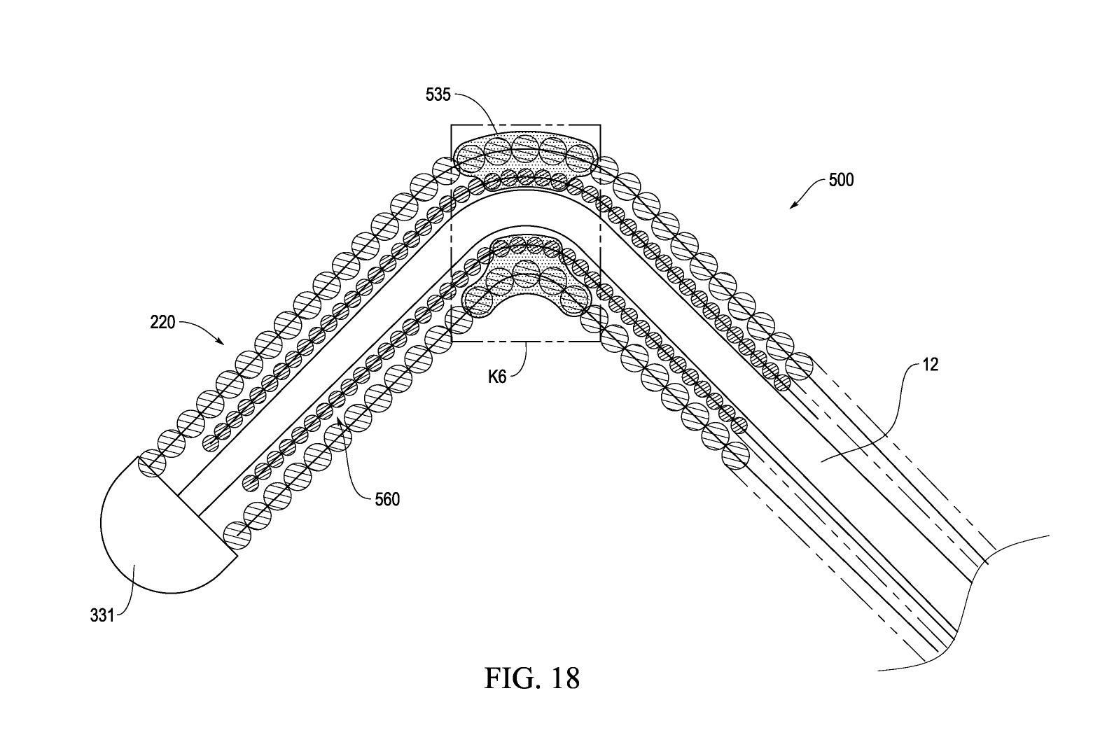

That is, the guide wire 500 also can be easily directed through tortuous blood vessels, and even if the distal end of the guide wire collides with a hard lesion, the guide wire 500 can alleviate the collision stress by allowing the stress to further reliably concentrate at the intermediate part of the coil body 220 (the part K6 at which the rigidity is enhanced by the joint portion 535) and by allowing the guide wire 500 to bend in substantially a V-shape (see FIG. 18). The stress does not progress proximally beyond the intermediate part of the first coil body 220, and plastic deformation of the shaft 12 can be suppressed more reliably. For simplicity, the detailed structures of the stranded wires 22 and 562 are not shown in FIG. 18.

What is claimed is:

1. A guide wire comprising:
a shaft;
a first coil body wound around a distal portion of the shaft, the first coil body being formed by helically winding a first stranded wire which is formed by winding a plurality of first element strands together; and
a joint portion disposed at an intermediate part of the first coil body so as to join at least two adjacent first stranded wires to each other without the joint portion contacting the shaft.

2. The guide wire of claim 1, wherein the first coil body is formed by helically winding a plurality of the first stranded wires.

3. The guide wire of claim 1, further comprising:
a second coil body disposed inside the first coil body, wherein the joint portion joins the first coil body and the second coil body to each other without contacting the shaft.

4. The guide wire of claim 2, further comprising:
a second coil body disposed inside the first coil body, wherein the joint portion joins the first coil body and the second coil body to each other without contacting the shaft.

5. The guide wire of claim 3, wherein the second coil body is formed by helically winding a plurality of second element strands.

6. The guide wire of claim 4, wherein the second coil body is formed by helically winding a plurality of second element strands.

7. The guide wire of claim 3, wherein the second coil body is formed by helically winding a plurality of second stranded wires, each second stranded wire being formed by winding a plurality of second element strands together.

8. The guide wire of claim 4, wherein the second coil body is formed by helically winding a plurality of second stranded wires, each second stranded wire being formed by winding a plurality of second element strands together.

9. The guide wire of claim 3, wherein the second coil body is formed by helically winding an element strand.

10. The guide wire of claim 4, wherein the second coil body is formed by helically winding an element strand.

11. The guide wire of claim 1, wherein a pitch of a helix of the first coil body is constant along a longitudinal direction of the guide wire.

12. The guide wire of claim 1, wherein the first coil body comprises a sparse winding section extending from the joint portion to a distal end of the first coil body and in which gaps between adjacent first stranded wires are greater than gaps between adjacent first stranded wires of the coil body extending from the joint portion to a proximal end of the first coil body.

13. The guide wire of claim 1, wherein the joint portion is formed of a joint material that is disposed between first element strands of the at least two adjacent first stranded wires.

14. The guide wire of claim 1, wherein the first stranded wire comprises a core element strand and a plurality of side element strands wound about a periphery of the core element strand.

15. The guide wire of claim 3, wherein the second stranded wire comprises a core element strand and a plurality of side element strands wound about a periphery of the core element strand.

16. The guide wire of claim 4, wherein the second stranded wire comprises a core element strand and a plurality of side element strands wound about a periphery of the core element strand.

17. A guide wire comprising:
a shaft;
a first coil body disposed around a distal portion of the shaft, the first coil body including a first stranded wire helically wound around the distal portion of the shaft, the first stranded wire including a plurality of first element strands that are wound together; and
a joint portion disposed at an intermediate part of the first coil body, the joint portion including a joint material that contacts and joins at least two adjacent windings of the first stranded wire to each other without the joint material contacting the shaft.
